(19) United States
(12) Patent Application Publication
(10) Pub. No.: US 2015/0107603 A1
(43) Pub. Date: Apr. 23, 2015

(54) RESERVOIR ANTECHAMBER FOR REDUCING FOAMING DURING SALIVA COLLECTION

(71) Applicant: APNICURE, INC., Redwood City, CA (US)

(72) Inventors: Jonathan L. PODMORE, San Carlos, CA (US); John Edwards CROWE, Menlo Park, CA (US); Nicholas R. VITALE, Foster City, CA (US); Michael C. Holzbaur, Menlo Park, CA (US)

(21) Appl. No.: 14/507,994

(22) Filed: Oct. 7, 2014

Related U.S. Application Data
(60) Provisional application No. 61/887,579, filed on Oct. 7, 2013.

(51) Int. Cl. A61C 17/06; A61M 1/00; A61F 5/56

(52) U.S. Cl. CPC A61C 17/046; A61F 5/566; A61M 1/0023; A61M 1/0001
USPC 128/847

(57) ABSTRACT

A saliva collector comprises a reservoir and a flow path from an inlet to an outlet on the reservoir. Air aspirated from the patient's oral cavity passes through an antechamber before reaching the reservoir. The antechamber is shaped to promote the collapse of any bubbles, allowing liquid saliva to drop from the antechamber to the reservoir.

### CROSS-REFERENCE

This application claims the benefit of U.S. Provisional Application No. 61/887,579 (Attorney Docket No. 41506-713.101), filed Oct. 7, 2013, which application is incorporated herein by reference.

The subject matter of this application is related that of co-pending U.S. patent application Ser. No. 14/297,811 (Attorney Docket No. 41506-712.201), filed on Jun. 6, 2014 and entitled “Heating Element for Reducing Foaming During Saliva Collection,” and U.S. Provisional Application No. 61/831,833 (Attorney Docket No. 41506-712.101), filed on Jun. 6, 2013 and also entitled “Heating Element for Reducing Foaming During Saliva Collection,” the full disclosures of which are incorporated herein by reference.

### BACKGROUND OF THE INVENTION

1. Field of the Invention

The present invention relates generally to medical devices and methods. In particular, the present invention relates to a reservoir and methods for its use for the collection of saliva with a reduction in bubbling and foaming.

A vacuum may be applied to an appliance or device held in a patient's oral cavity for a variety of purposes. For example, an appliance for treating obstructive sleep apnea (OSA) may utilize a device held in a patient's mouth where a vacuum is constantly drawn on the device in order to reposition portions of the patient's oral anatomy to reduce the likelihood of OSA. The device may be used for or in conjunction with drawing a patient's tongue and/or lower mandible forward in order to reduce OSA. Of particular interest to the present invention, the vacuum may be drawn in order to help draw the soft palate and/or rear portion of a patient's tongue away from the pharynx in order to maintain a clear breathing passage.

In all such devices which draw a partial negative pressure within the oral cavity, there is a likelihood that a flow of saliva will be created in tubes and other flow passages connected to the oral appliance to maintain the vacuum. In order to avoid fouling the equipment which produces the vacuum, a saliva collector may be provided in-line to remove and collect the saliva.

FIG. 1 is taken from FIG. 25 of co-pending, commonly owned U.S. Patent Publication No. 2012/0132216, the full disclosure of which is incorporated herein by reference. FIG. 1 illustrates a system 489 including an oral device 490, a vacuum pump 492, a saliva reservoir 494, and a pressure sensor 496. Oral device 490 further includes a pressure conduit 498 extending through bite structure 500 to the superior side of tongue constraint 502 where pressure conduit 498 has a distal opening 504. The pressure conduit 498 may alternatively comprise an inner lumen formed integrally within tongue constraint 502 or bite structure 500, and distal opening 504 could be positioned in any of various positions relative to bite structure 500 as may be desired to measure pressure within the oral cavity. A vacuum lumen 506 extends from the superior surface of tongue constraint 502 through bite structure 500 and both vacuum lumen 506 and pressure conduit 498 extend through lip seal 508. Vacuum lumen 506 is connected to a vacuum tube 510 which connects in an airtight manner to an input fitting 512 on saliva reservoir 494. Vacuum tube 510 has a vent hole 511 anterior to lip seal 508 so as to be outside the patient's oral cavity but positioned as close to oral device 490 as practicable while minimizing risk of obstruction by the patient's lips or other tissues. Alternatively, vent hole 511 may be disposed in vacuum lumen 506 anterior to bite structure 500 or on the superior side of tongue constraint 502 so as to be located within the patient's oral cavity. When a negative pressure (partial vacuum) is applied through vacuum lumen 506 within the patient's oral cavity, saliva or other liquids which collect may be aspirated through vacuum lumen 506 and vacuum tube 510. While removing excess liquids from the oral cavity is desirable, the weight of the liquid within vacuum tube 510 may create a pressure offset in vacuum tube 510 which would then affect the negative pressure applied within the oral cavity. System 489 alleviates this problem by providing vent hole 511 in vacuum tube 510, allowing any aspirated liquids to flow to saliva reservoir 494 more quickly.

While effective, the saliva collection system described above can result in the mixing of air and saliva in the vacuum flow path which in turn will create bubbles and foam inside of the reservoir. In some cases, it is possible for the bubbles and foam to accumulate so that they reach the outlet fitting 516 connected to vacuum pump 492, as illustrated in FIG. 1. Even if the foaming does not reach that level, handling and/or accidental disturbance of the reservoir in such systems can allow saliva to enter the vacuum tubing leading to the vacuum pump.

For these reasons, it would be desirable to provide alternative and improved methods and apparatus for removing and collecting saliva in vacuum systems used with oral appliances and other devices. The methods and devices should be effective in cases of even the most excessive bubbling and foaming as well as in cases where the reservoir may be completely inverted. Such methods and systems should be simple and inexpensive to implement. At least some of these objectives will be met by the inventions described hereinafter.

2. Description of the Background Art

U.S. Patent Publication No. 2012/0132216 has been described above. U.S. Ser. No. 61/831,833, filed on Jun. 6, 2013, U.S. Ser. No. 13/546,453, filed on Jul. 11, 2012, and U.S. Ser. No. 13/023,763, filed on Feb. 9, 2011, the full disclosures of which are incorporated herein by reference, are co-pending, commonly owned U.S. Patent Applications and describe alternative saliva management systems of oral appliances. Oral and external devices for treating sleep apnea and snoring are described in U.S. Patent Publication Nos. US2005/166929; US2005/166928; US2008/0188947; US2007/0277818; US2008/0216843; and US2008/0210244; and in U.S. Pat. Nos. 7,182,082; 7,073,506; 7,073,505; 6,955,172; 6,877,513; 6,494,209; 5,957,133; 5,465,734; 4,676,240; 4,304,227; 4,169,473; and 3,132,647.

### SUMMARY OF THE INVENTION

The present disclosure provides apparatus and methods for the improved collection of saliva from aspirated air streams entrained with saliva, typically originating from a patient's oral cavity. In particular, the present disclosure provides for collecting saliva with reduced or eliminated formation of bubbles and foam in a collection reservoir. As described above, use of a vacuum to aspirate air from a patient's oral cavity can result in entrained saliva which should be removed before the aspirated air stream reaches a vacuum pump or other vacuum source. While a simple collection reservoir may be placed in a vacuum line from the oral cavity before the pump, as described in U.S. Patent Publication No. 2012/0132216, where the majority of saliva will drop to the bottom of the collection reservoir, excessive bubbles and foaming can result in loss of saliva through an outlet port on the reservoir, thus risking saliva reaching the vacuum pump or other vacuum source.

While the passage of saliva bubbles and foam through the outlet port might be overcome by a simple membrane or other barrier placed over the outlet port, it has been found by the inventors herein that such a simple barrier can itself become fouled over time which can interfere with operation of the vacuum system. Thus, even if saliva is inhibited from leaking from the reservoir, operation of the vacuum system may still be impaired.

The present disclosure provides for further improvement in saliva collection reservoirs and methods by placing a heating element alone or more usually in tandem with a membrane along an air flow path from the oral device to the vacuum or other source. In particular, the heating element applies localized heat to break bubbles formed in the air aspirated from the oral cavity of the patient or suppress the formation of the bubbles. Heat is applied to evaporate the fluid wall of the bubbles. The heating element can be positioned anywhere along the flow path, including adjacent an outlet port into the saliva collection reservoir, adjacent a membrane or other barrier placed over the outlet port, and within the interior volume of the reservoir. The heating element may comprise a resistive wire or a ceramic heating element. The saliva resulting from the suppression and breakage of bubbles and foam drains to be collected at the bottom of the reservoir or simply evaporates.

The present disclosure also provides for further improvement in saliva collection reservoirs and methods by providing an antechamber along an air flow path from the oral device to the vacuum or other source. In particular, air aspirated from the oral cavity of a patient passes through the antechamber before entering the interior volume of the reservoir. The geometry of the antechamber is configured to encourage bubble popping. The shape of the antechamber and its multiple openings to the interior volume of the reservoir stress the bubbles in a non-uniform way and encourages the breakage of the bubbles. The saliva resulting from the suppression and breakage of bubbles and foam drains from the antechamber to be collected at the bottom of the reservoir.

An aspect of the present disclosure provides a saliva collector for attachment in a vacuum line which aspirates an air stream entrained with saliva. The saliva collector comprises a reservoir and a heating element. The reservoir has a bottom, a top, and a sidewall which together define an interior volume. The reservoir further has an air inlet and an air outlet with an air flow path therebetween. The heating element disrupts bubbles and foam present in the air stream such that the disrupted bubbles and foam evaporate or drain into the interior volume of the reservoir as liquid saliva or some combination of both. Typically, the heating element is positioned along the air flow path within the reservoir but may be positioned along the path of the air stream before the reservoir, alternatively or in combination.

The saliva collector may further comprise a membrane positioned within the interior of the reservoir on the flow path so that all air passes therethrough before passing through the outlet. Exemplary membranes that can be used are described in co-owned and co-pending U.S. patent application Ser. No. 13/546,453, filed on Jul. 11, 2012, the disclosure of which is fully incorporated herein by reference. The membrane can permit the flow of air but can block the passage of saliva. The heating element may be positioned adjacent the membrane. The bubble barrier may comprise a mesh which may comprise one or more resistive wires that can be heated. The bubble barrier may comprise a perforate barrier.

In the exemplary embodiments, the bubble barrier will be a cylindrical mesh or perforated wall which is arranged axially within the reservoir to define an outer annular region for receiving the airflow from the patient's oral cavity and an inner region which allows fluid collection and flow of the pre-treated air from which the bubbles and foam have been removed. The use of such a vertical, cylindrical barrier can be advantageous since it can maximizes the area available to disrupt the foam and bubbles and is least affected by a rising level of the saliva as it drains and collects on the bottom of the reservoir.

In the exemplary embodiments, where the bubble barrier is a cylinder, the outer side wall of the reservoir will preferably also be cylindrical, thus forming an outer annular region within the reservoir for receiving the untreated air and an inner cylindrical region for allowing the pre-treated air to flow upwardly to the barrier and the outlet port. In exemplary embodiments, the reservoir will have a volume in the range from about 10 cm³ to 1000 cm³, and the bubble barrier will have a surface area of 20 cm² to 200 cm².

The heating element may be positioned adjacent one or more of the inlet or the outlet. The heating element may comprise one or more of a resistive wire or a ceramic heating element. The resistive wire may comprise a nickel chromium wire. The heating element can be configured to provide heat at a temperature sufficient to evaporate liquid walls of saliva bubbles. For example, the heating element can be configured to be heated to a temperature of at least 100° C. and in some cases up to 250° C. or any other viable threshold. The heating element can be configured to apply heat at intervals. The saliva collector may further comprise a temperature sensing element operatively coupled to the heating element. The temperature sensing element can be configured to turn off the heating element once the heating element has reached a threshold temperature.

The saliva collector may further comprise one or more of a current sensing element, a resistance sensing element, or an impedance sensing element operatively coupled to the heating element. The current, resistance, or impedance sensing element(s) may be configured to detect the presence of bubbles and foam near the heating element. The current, resistance, or impedance sensing element(s) may be configured to adjust the power of the heating element in response to the detected presence of bubbles and foam near the heating element. For example, the current, resistance, or impedance sensing element(s) may detect dips in current that may indicate the presence of one or more bubbles and activate the heating element when one or more bubble are detected.

Exemplary embodiments of the present disclosure will further comprise inlet and outlet valves at the inlet and outlet of the reservoir, respectively. The valves will typically be self-opening valves which open when a line or fitting are connected to the reservoir for use and which close when the line or fitting is removed. In this way, the reservoir can be conveniently removed from the system while minimizing the risk that the collected saliva will be unintentionally spilled.

The top of the reservoir may comprise an antechamber positioned along the air flow path. The antechamber can have an interior volume shaped to promote the collapse of bubbles and foam present in the air stream before such bubbles or foam can exit the outlet. Embodiments of the saliva collector reservoir may use one or more of the heating element, the membrane, or the antechamber to disrupt bubbles and foam present in the air stream.

Another aspect of the present disclosure provides a method for removing saliva from an air stream aspirated from a patient's oral cavity. The air stream is directed through a reservoir from an inlet, along a flow path, and to an outlet. Saliva entrained in the air stream can form bubbles and foam. The air stream is passed through a heating element positioned along the flow path of the air stream to cause bubbles and foam to collapse such that the collapsed bubbles and foam evaporate or drain into an interior volume of the reservoir as liquid saliva or some combination of both. Typically, the heating element is positioned along the air flow path within the reservoir but may be positioned along the path of the air stream before the reservoir, alternatively or in combination.

In many embodiments, the pre-treated air stream is passed through a membrane to separate the entrained liquid saliva. The heating element can be positioned adjacent the membrane.

The air stream can be directed by drawing a partial vacuum on the outlet of the reservoir, typically a vacuum in the range from 2 cm H₂O to 250 cm H₂O. The air stream in many cases originates from an oral appliance held in the patient's oral cavity. The oral appliance may be connected to the inlet of the reservoir by tubing. The flow rate of the air stream will typically be in the range from 20 ml/min to 1000 ml/min.

The reservoir may be disconnected from inlet and outlet conduits, the collected saliva may be drained, the heating element may be cleaned, and the reservoir may be reconnected to the inlet and outlet conduits.

The heating element can be positioned adjacent one or more of the inlet and the outlet. The heating element can comprise one or more of a resistive wire or a ceramic heating element. The heating element can be heated to a temperature sufficient to evaporate liquid walls of saliva bubbles to cause the bubbles and foam to collapse. For example, the heating element can be heated to a temperature of at least 100° C. and in some cases up to 250° C. or any other viable threshold. The heat can be applied at intervals. The temperature of the heating element can be measured and the heating element may be turned off once the heating element has reached a threshold temperature. The pre-treated air stream can be passed through an antechamber along the flow path. The antechamber can have an interior volume shaped to promote the collapse of bubbles and foam present in the air stream before such bubbles and foam can exit from the outlet. Embodiments of the saliva collector reservoir may use one or more of the heating element, the membrane, or the antechamber to disrupt bubbles and foam present in the air stream.

One or more of a current, a resistance, or an impedance of the heating element may be measured. The presence of bubbles and foam near the heating element may be detected in response to the measurement of the one or more of the current, resistance, or impedance of the heating element. A power of the heating element can be adjusted in response to the detection of the presence of bubbles and foam near the heating element. For example, dips in current detected as changes in current, resistance, or impedance may indicate the presence of one or more bubbles, and subsequently the power of the heating element may be adjusted to disrupt the one or more bubbles.

A further aspect of the present disclosure provides a saliva collector for attachment in a vacuum line which aspirates an air stream entrained with saliva. The saliva collector comprises a reservoir having a bottom, a top, and a sidewall which together defines an interior volume. The reservoir further has an air inlet and an air outlet with an air flow path therebetween. The top of the reservoir comprises an antechamber positioned along the air flow path. The antechamber has an interior volume shaped to promote the collapse of bubbles and foam present in the air stream before such bubbles or foam can exit from the outlet. The air inlet may be oriented transverse to gravity to guide the aspirated air to the interior volume of the antechamber. The air inlet can have a variety of shapes including, but not limited to, a circle, an oval, an ellipse, a triangle, a square, and combinations thereof. The antechamber can comprise one or more openings configured to allow collapsed bubbles and foam to drop or drain into the interior volume of the reservoir. These opening(s) may open downward toward the interior volume of the reservoir. Typically, the saliva collector further comprises a barrier or membrane separating the reservoir into an outer reservoir portion and an inner reservoir portion. The barrier or membrane generally has a cylindrical shape. The air flow crosses the barrier or membrane which can disrupt saliva bubbles in the air flow. The antechamber for promoting the collapse of bubbles and foam present in the air stream can be used alone to promote such collapse or be used in combination with one or more of a heating element or membrane to further promote such collapse.

A further aspect of the present disclosure provides a method for removing saliva from an air stream aspirate from a patient's oral cavity. The air stream is directed through a reservoir from an inlet, along a flow path, and to an outlet. Saliva entrained in the air stream can form bubbles and foam. The air stream is passed through an antechamber positioned along the flow path to cause bubbles and foam to collapse to provide a pre-treated air stream before passing out through the outlet. The antechamber has an interior volume shaped to promote the collapse of bubbles and foam present in the air stream. The air inlet can be oriented transverse to gravity to guide the aspirated air to the interior volume of the antechamber. The air inlet can have a variety of shapes including, but not limited to, a circle, an oval, an ellipse, a triangle, a square, and combinations thereof. Collapsed bubbles and foam can be allowed to drop or drain into the interior volume of the reservoir through one or more openings of the antechamber. These opening(s) may open downward toward the interior volume of the reservoir. Typically, the saliva collector further comprises a barrier or membrane separating the reservoir into an outer reservoir portion and an inner reservoir portion. The barrier or membrane generally has a cylindrical shape. The air flow crosses the barrier or membrane which can disrupt saliva bubbles in the air flow. The antechamber for promoting the collapse of bubbles and foam present in the air stream can be used alone to promote such collapse or be used in combination with one or more of a heating element or membrane to further promote such collapse.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates a prior art system as found in US Patent Application No. 2012-0132216;

FIG. 2 is a flow chart illustrating the air stream flow and treatment steps of the methods of the present disclosure;

FIGS. 3A and 3B are schematic illustrations of the first saliva collection reservoir system of the present disclosure;

FIGS. 4A and 4B illustrate a more detailed second saliva collection reservoir system of the present disclosure;

FIG. 5 is a top plan view of a portion of the saliva collection reservoir of FIGS. 4A and 4B;

FIG. 6 is a cross-sectional view taken along line 6-6 of FIG. 5;

FIG. 7 is a flow chart illustrating the air stream flow and treatment steps of the methods of the present disclosure in which a heating element is used;

FIG. 8 is a cut-away view of a saliva collection reservoir system comprising one or more heating elements, according to embodiments of the disclosure;

FIG. 9A is a perspective view of the bottom of a saliva collection reservoir top comprising a heating element arranged in a spiral pattern, according to embodiments of the disclosure;

FIG. 9B is a perspective view of the bottom of a saliva collection reservoir top comprising a heating element arranged in a zig-zag pattern, according to embodiments of the disclosure;

FIG. 10 is a flow chart illustrating the air stream flow and treatment steps of the methods of the present disclosure in which a bubble and foam collapsing antechamber is used;

FIG. 11 is a perspective view of the bottom of a saliva collection reservoir top comprising a bubble and foam collapsing antechamber, according to embodiments of the disclosure;

FIG. 12 is a cut-away view of a saliva collection reservoir system comprising the top of FIG. 11, according to embodiments of the disclosure;

FIG. 13 is a bottom view of another saliva collection reservoir top comprising a bubble and foam collapsing antechamber, according to embodiments of the disclosure;

FIG. 14A is a sectional view of the saliva collection reservoir of FIG. 13 taken from line 140 in FIG. 13 wherein the air inlet has a circular shape, according to embodiments of the disclosure;

FIG. 14B is a sectional view of the saliva collection reservoir of FIG. 13 taken from line 140 in FIG. 13 wherein the air inlet has an oval shape, according to embodiments of the disclosure; and

FIG. 14C is a sectional view of the saliva collection reservoir of FIG. 13 taken from line 140 in FIG. 13 wherein the air inlet has a rectangular shape, according to embodiments of the disclosure.

### DETAILED DESCRIPTION OF THE INVENTION

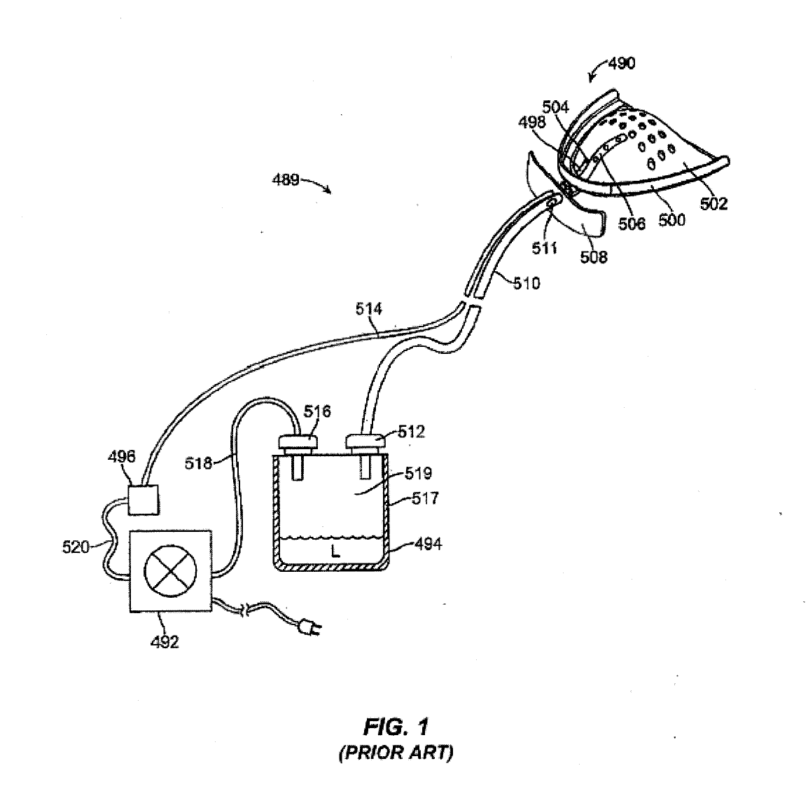

The saliva collectors and reservoirs of the present disclosure may be used in a variety of systems, typically systems where a vacuum line is being used to withdraw an air stream from a patient's oral cavity. Exemplary of such systems is system 489 illustrated in FIG. 1 where the reservoirs of the present invention might be used in place of conventional saliva reservoir 494.

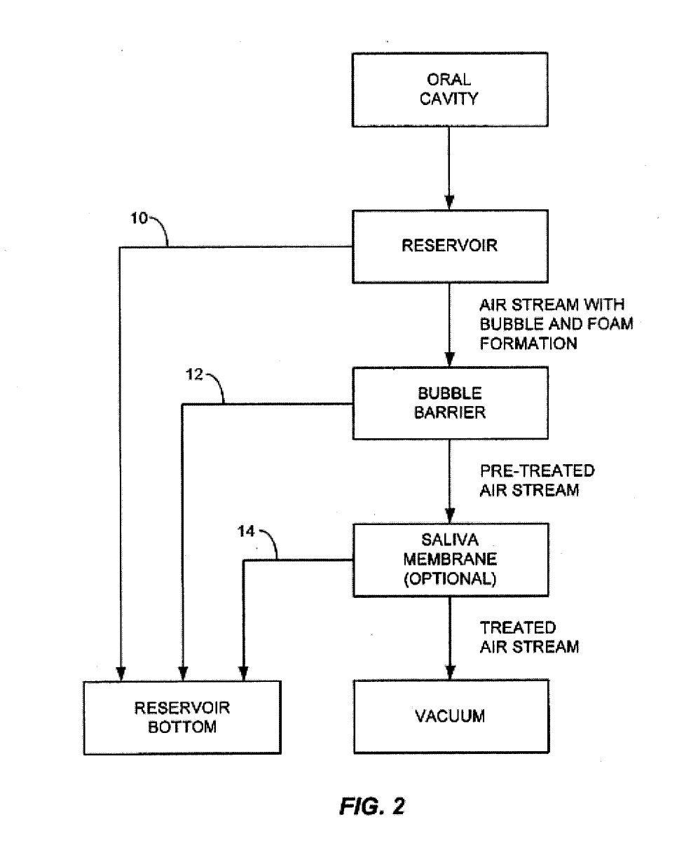

Referring to FIG. 2, the apparatus and methods of the present disclosure provide for drawing an air stream from an oral cavity using a vacuum source, such as a pump. The air stream first passes into a reservoir where a first volume 10 of saliva separates by gravity and falls to the reservoir bottom. The remaining air stream will typically have entrained bubbles and saliva foam which is to be removed before the air stream reaches a saliva membrane to remove entrained liquid saliva. The removal of the bubbles and foam is accomplished with a bubble barrier to produce a pre-treated air stream which is then directed through the saliva membrane. The treated air stream leaving the saliva membrane will then be directed out of the reservoir and flow directly or indirectly to the vacuum pump or other source. A quantity or volume 12 of liquid saliva resulting from disruption of the bubbles and foam by the bubble barrier will also drop to the reservoir bottom as will a third volume or quantity of 14 of liquid saliva which is produced by the saliva membrane.

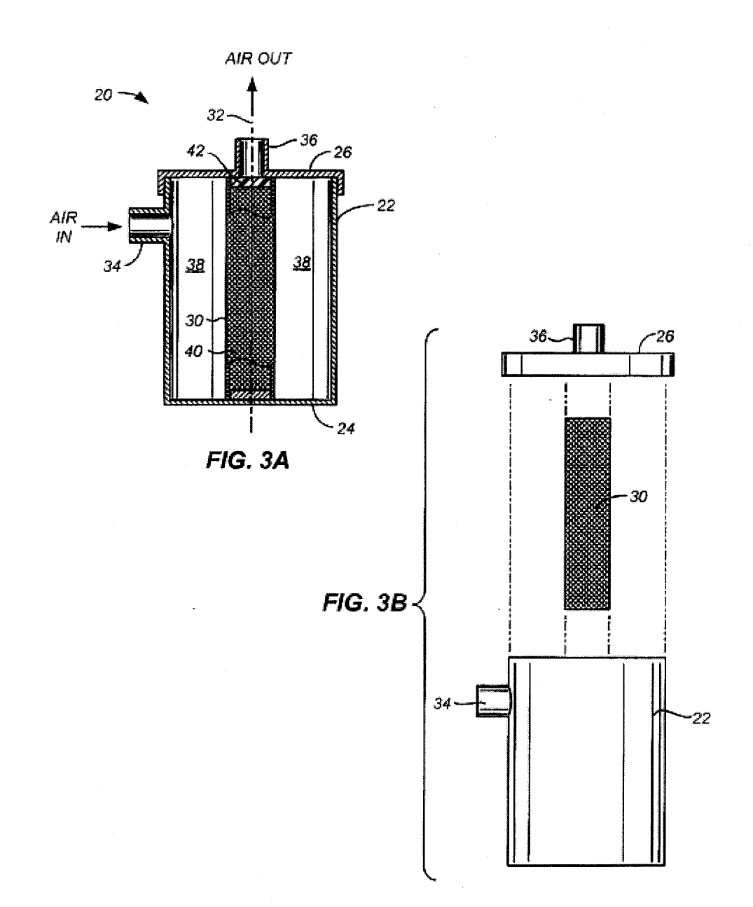

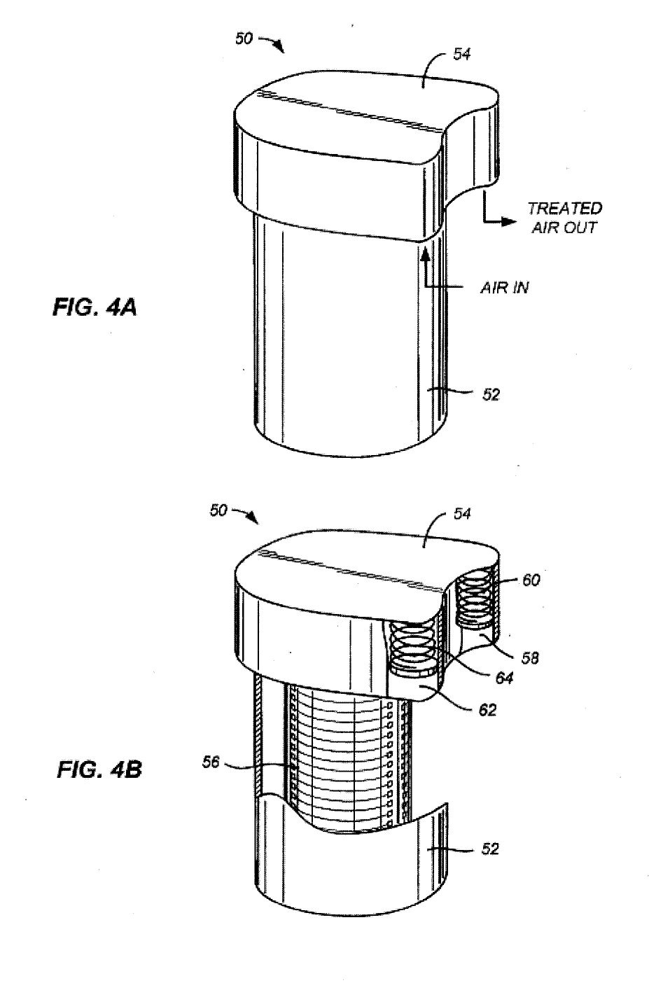

Referring to FIGS. 3 and 3B, a saliva collection reservoir 20 constructed in accordance with the principles of the present invention will include a reservoir enclosure 22 having a bottom 24, a removable top 26, and a cylindrical side wall 28. A bubble barrier 30, in the form of a cylindrical mesh or perforated wall, is aligned centrally along a vertical axis 32 of the reservoir body 22. An inlet port 34 is provided in the side wall of the body 22, typically near the top, and an outlet port 36 is formed centrally in the removable top 26 so that it is coaxially aligned with axis 32. In this way, an interior of the reservoir body 22 is divided into an outer, annular volume 38 and an inner cylindrical volume 40 (located within the cylindrical bubble barrier 30). Thus, air having entrained liquid saliva, foam, and bubbles entering through inlet port 34 will first enter and circulate around the annular volume 38 where liquid saliva will be able to separate and drop to the bottom of the reservoir. Before entering the inner cylindrical volume 40, however, the air will have to pass through the perforations of the bubble barrier 30, where the perforations will disrupt foam and bubbles which may be present. The foam and bubbles will be physically disrupted so that they coalesce and return to the liquid state, separate, and fall to the bottom of the reservoir. The pre-treated air stream which flows from the bubble barrier 30 into the inner cylindrical volume 40 will thus be free of entrained bubbles and foam, but will still have entrained liquid saliva which will be carried to the saliva membrane 42 before the air can exit through outlet 436. The saliva membrane 42 will separate the liquid saliva before the saliva can reach the vacuum pump. FIG. 3B shows the components of the saliva collection reservoir 20 in an exploded view. Referring now to FIGS. 4A and 4B, a second embodiment of a saliva collection reservoir 50 will be described. The saliva collection reservoir 50 includes the same basic components as reservoir 20, but further includes inlets and outlets having self-opening and closing valves so that the reservoir may be removed from a vacuum line with reduced risk of spillage.

The saliva collection reservoir 50 includes a cylindrical canister 52 and a removable top 54. A cylindrical perforate barrier 56 is axially aligned within the anterior of the cylindrical canister 52, and an outlet 58 having an outlet valve 60 and an inlet 62 having an inlet valve 64 are disposed in the removable top 54.

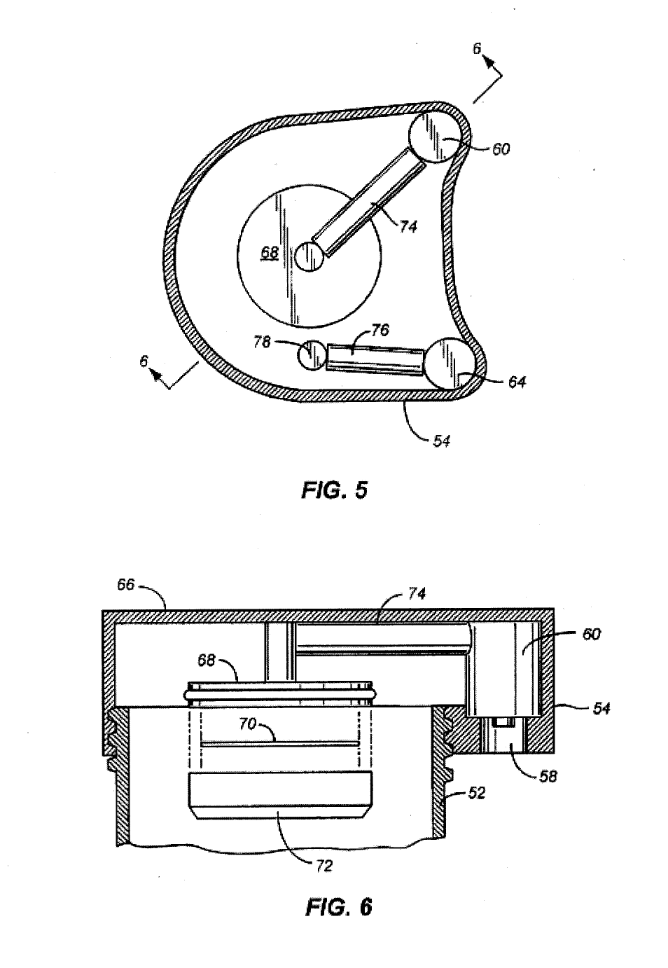

More detailed construction of the interior of the saliva collection reservoir 50 and of the flow paths therein are seen in FIGS. 5 and 6. FIG. 5 is a plan view of removable top 54 with the very top plate 66 (FIG. 6) removed. A fitting 68 attached within the removable top 54 receives the saliva membrane 70, which is held in place by a retaining ring 72. The retaining ring engages the cylindrical perforated barrier (FIG. 4B), so that the pre-treated air stream flows upwardly through the barrier into fitting 68 and then radially outwardly through tube 74 to the valve 60 and outlet 58. As best seen in FIG. 5, the inlet air passes in through valve 64, and inwardly through connecting tube 76, and then to a port 78, which passes the inlet air stream into the outer annular volume of the cylindrical canister 52.

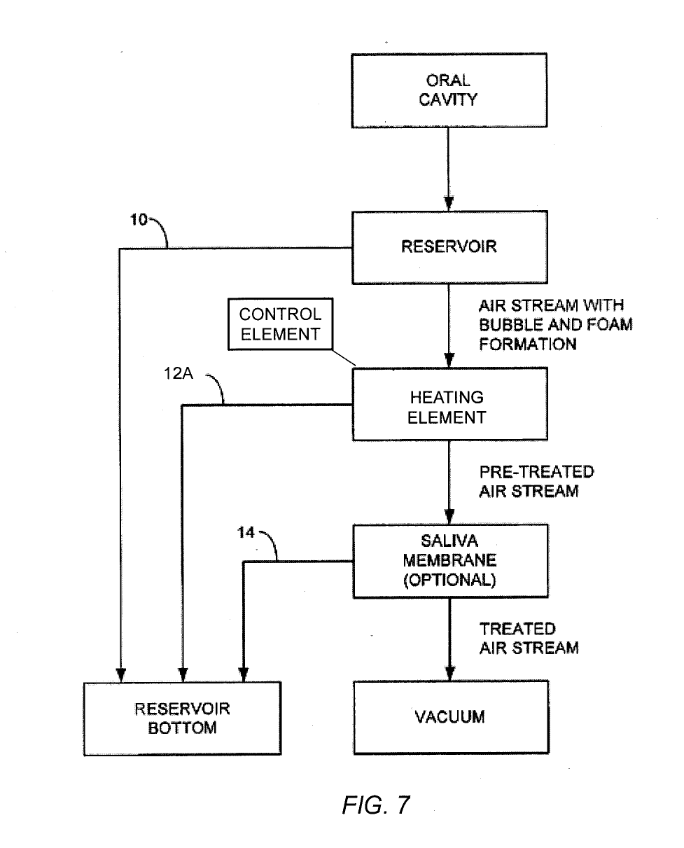

Referring to FIG. 7, further embodiments of the apparatus and methods of the present disclosure provide for drawing an air stream from an oral cavity using a vacuum source, such as a pump. The air stream first passes into a reservoir where a first volume 10 of saliva separates by gravity and falls to the reservoir bottom. The remaining air stream will typically have entrained bubbles and saliva foam which is to be removed before the air stream reaches a saliva membrane to remove entrained liquid saliva. The removal of the bubbles and foam is accomplished with a heating element to produce a pre-treated air stream which is then directed through the saliva membrane. The heating element can provide heat to evaporate the liquid walls of saliva bubbles. The heating element can comprise a resistive wire such as a nickel chromium wire, although other materials or other types of heating elements such as ceramic heaters may be used alternatively or in combination. The treated air stream leaving the saliva membrane will then be directed out of the reservoir and flow directly or indirectly to the vacuum pump or other source. A quantity or volume 12A of liquid saliva resulting from disruption of the bubbles and foam by the heating element will also drop to the reservoir bottom as will a third volume or quantity of 14 of liquid saliva which is produced by the saliva membrane. Alternatively or in combination, a heating element may be provided along the path of the air stream before the reservoir, for example, to apply heat to the air stream to prevent the formation of bubbles and foam.

In many embodiments, a control element operatively coupled to the heating element is provided. The control element may be configured to sense one or more of the current, resistance, or impedance of the heating element. The detected current, resistance, or impedance can indicate the presence of bubbles and foam near the heating element. The control element may adjust the power of the heating element in response to the detected presence of bubbles and foam near the heating element. For example, detected dips in current that may indicate the presence of one or more bubbles and the heating element can be activated when one or more bubble are detected. In particular, the current, resistance, or impedance of the heating element is monitored while running the wire at a fixed, but low, voltage such that fluctuations in the measured parameters are expected when fluid or bubbles come in contact with the heating element. The control element may turn up the current briefly in response to such fluctuations to pop some bubbles before returning the current to the base, monitoring level until the next bubbles present themselves. Alternatively or in combination, the control element detects the temperature of the heating element and may turn the heating element off or adjust its power when the heating element has reached a threshold temperature.

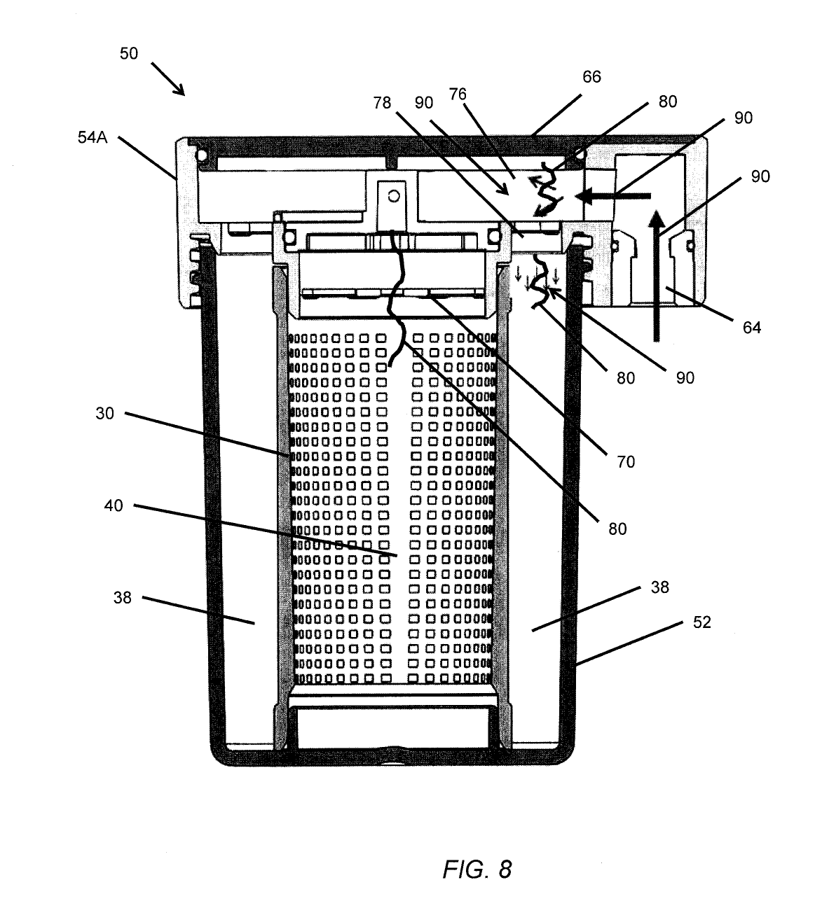

Referring to FIG. 8, the saliva collection reservoir 50 may further comprise one or more heating elements 80 for applying heat to disrupt bubbles and foam from aspirated saliva. The heating element 80 may comprise one or more resistive wires, such as nickel chromium wires, that apply heat to evaporate the liquid walls of saliva bubbles and foam as the aspirated air and saliva flows through the flow path 90. The saliva collection reservoir 50 comprises a removable top 54A which may be similar to removable top 54 described above. The removable top 54A further comprises one or more heating elements 80 along the flow path 90. The flow path 90 can be similar to the flow path for the removable top 54 described above. The aspirated air and saliva enters the saliva collection reservoir 50 through the inlet valve 64, and inwardly through connecting tube 76, and then to a port 78, which passes the inlet air stream into the outer annular volume 38 of the cylindrical canister 52.

The heating element 80 can be positioned to prevent saliva bubbles from fouling the saliva membrane 80 or from exiting the reservoir 50 in the absence of the membrane 80. The heating element 80 can be positioned in many locations along the flow path 90. As shown in FIG. 8, the heating element 80 can be positioned adjacent one or more of the inlet valve 64, the connecting tube 76, the port 78, the saliva membrane 70, or anywhere such that the saliva bubbles and foam would not reach the exit of the reservoir and pass into the console such as the vacuum pump 492. In exemplary embodiments, the heating element 80 is positioned in from of the small outflow port 78 of the reservoir 50. Alternatively or in combination, the heating element 80 can be positioned in front of the saliva membrane 80. Alternatively or in combination, the heating element 80 can be positioned immediately past the reservoir inlet valve 64. Alternatively or in combination, the heating element 80 can be integrated into the bubble barrier or mesh 30.

The heating element 80 can have any number of shapes or configurations. The heating element 80 may comprise a single wire filament crossing the flow path 90. Alternatively, the heating element 80 may comprise a plurality of wire filaments driven in parallel. In some embodiments, the filament(s) may be shaped to cross back and forth across the flow path 90 one or more times. In some embodiments, the filament(s) may comprise one or more conical coils, one or more spiral flat windings, or the like.

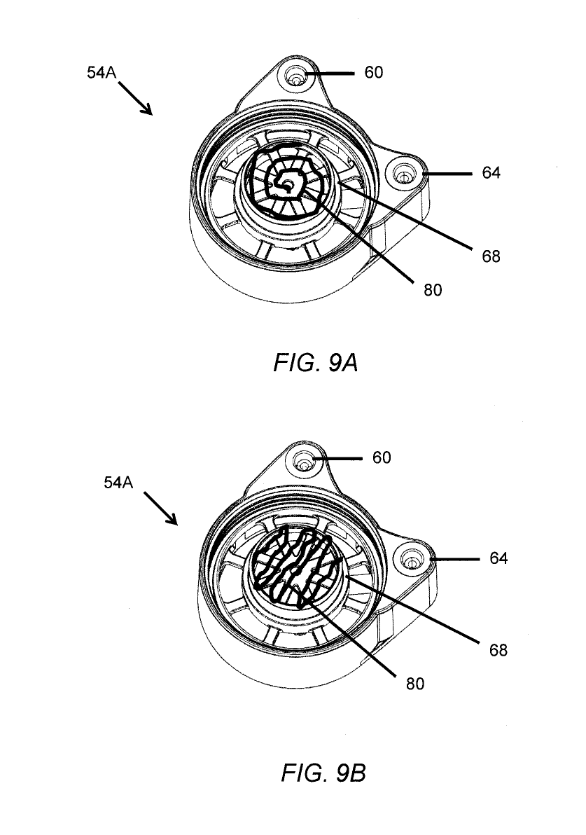

Referring to FIGS. 9A and 9B, the reservoir top 54A may comprise one or more heating elements 80. The heating element 80 may be positioned adjacent the fitting 68 to be positioned near a saliva membrane 70 of the reservoir 50 when assembled. The heating element 80 can comprise a resistive wire that is heated by conduction, for example, a nickel chromium wire. As shown in FIG. 9A, the heating element 80 may comprise a wire arranged in a spiral pattern. As shown in FIG. 9B, the heating element 80 may comprise a wire arranged in a zig-zag pattern.

In an exemplary embodiment, the heating element 80 comprises a nickel chromium wire through which a current is driven through. For example, a current of 400-500 mA can be driven through the nickel chromium wire to pop bubbles. In one experimental example, an applied voltage of 1.15 V can generate a current of 400 mA through the nickel chromium wire to generate sufficient heat to evaporate fluid and pop a bubble within one or two seconds of contact. In another experimental example, a voltage of 1.28 V can generate a current of 500 mA through the nickel chromium wire to generate sufficient heat to evaporate fluid and pop a bubble immediately upon contact. The heat applied by the heating element may be at least 100° C. For example, heat at a temperature of at least 100° C. may be enough to quickly pop a saliva bubble without too much residence time in contact, but higher temperatures may provide further advantages in at least some instances. In another example, the heating element 80 can be heated to a temperature of up to 250° C. In some embodiments, the heat may be applied in pulses or at intervals. In some embodiments, heat may be applied by the heating element 80 in conjunction with temperature sensing of the heating element, for example, to determine whether there is fluid in contact with the heating element 80.

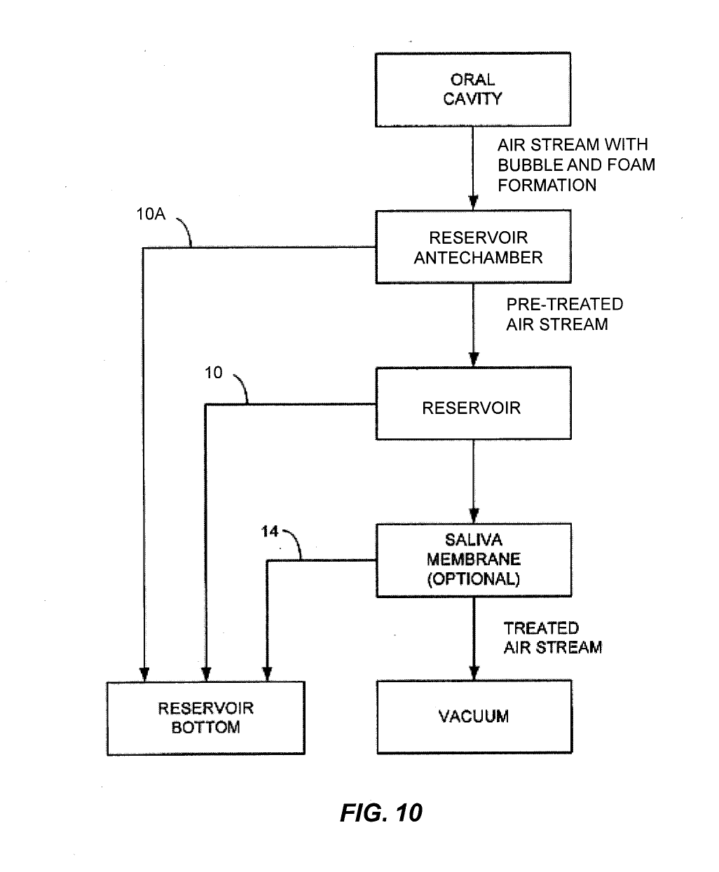

Referring to FIG. 10, yet further embodiments of the apparatus and methods of the present disclosure provide for drawing an air stream from an oral cavity using a vacuum source, such as a pump. The air stream first passes into a reservoir antechamber having an internal volume shaped to promote the disruption of bubbles and foam from the air aspirate. A volume 10A of the liquid saliva from the disrupted bubbles and foam falls to the reservoir bottom. The air stream then passes into a reservoir where a volume 10 of saliva separates by gravity and falls to the reservoir bottom. The remaining air stream will typically have entrained bubbles and saliva foam which is to be removed before the air stream reaches a saliva membrane to remove entrained liquid saliva. The treated air stream leaving the saliva membrane will then be directed out of the reservoir and flow directly or indirectly to the vacuum pump or other source. A quantity or volume 14 of liquid saliva produced by the saliva membrane will drop to the reservoir bottom.

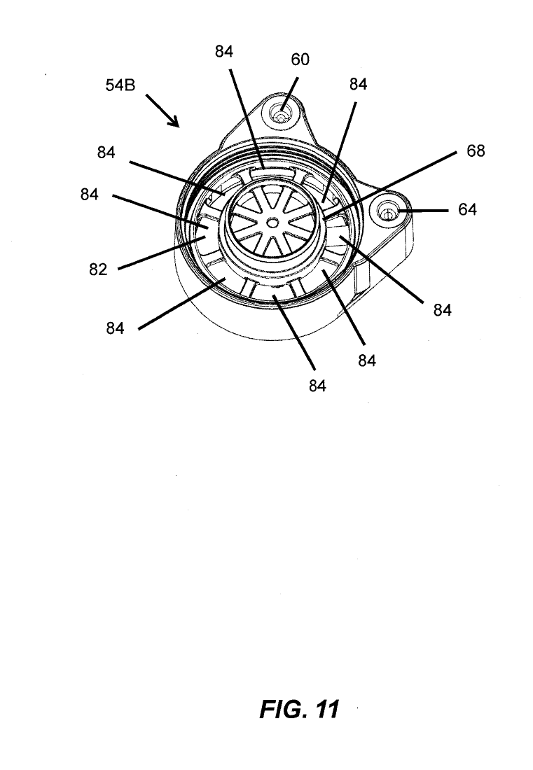

FIG. 11 is a perspective view of the bottom of a saliva collection reservoir top 54B comprising a bubble and foam collapsing antechamber 82. The antechamber 82 has a plurality of openings 84. The antechamber 82 has an internal geometry shaped to promote the disruption of foam and bubbles. When the saliva collection reservoir 50 is assembled with the top 54B, the openings allow liquid saliva to drop into the reservoir bottom.

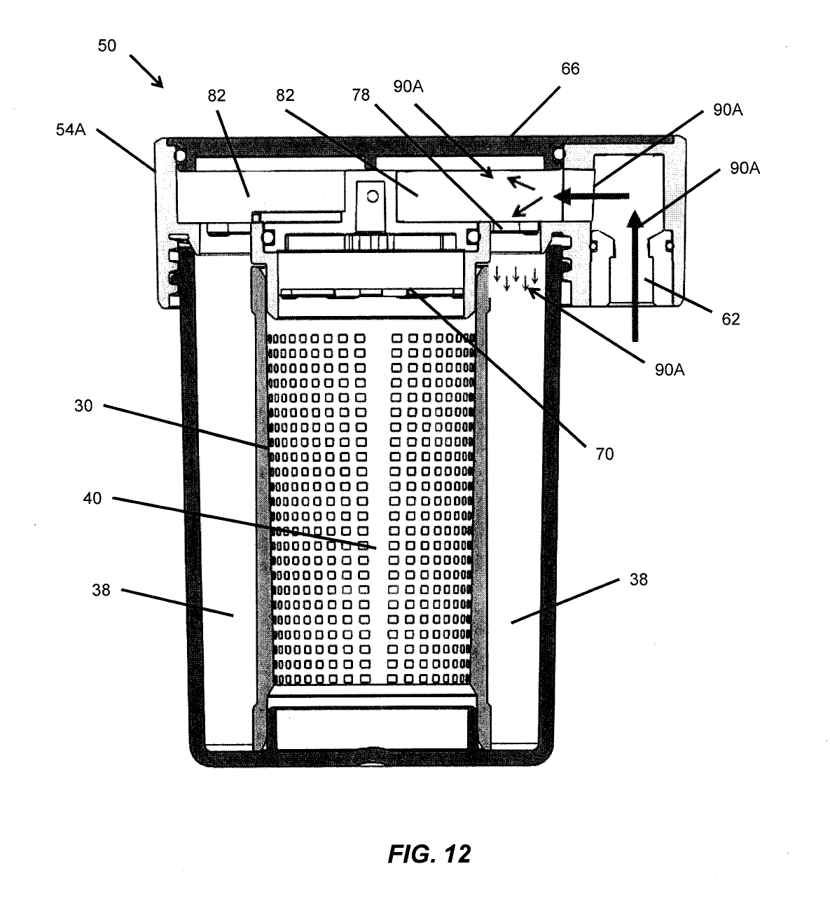

FIG. 12 is a cut-away view of a saliva collection reservoir system 50 assembled with the top 54B. FIG. 12B also shows the flow path 90A of air and saliva aspirated from a subject. The flow path 90A can be similar to the flow path 90 described above. The aspirated air and saliva enters the saliva collection reservoir 50 through the inlet valve 64 and inwardly through the inner volume of the antechamber 82, which passes the inlet air stream into the outer annular volume 38 of the cylindrical canister 52 through openings 84. In many embodiments, one or more of the inlet valve 64 or the inlet opening into the antechamber 84 can have a round or non-round shape perpendicular or at least transverse to gravity. This inlet orientation can result in bubbles and foam being blown into the antechamber 82. In many embodiments, one or more of the shape of the antechamber 82 and the plurality of openings 84 of the antechamber can stress the bubbles and foam in a non-uniform way to result in breakage of the bubbles and foam.

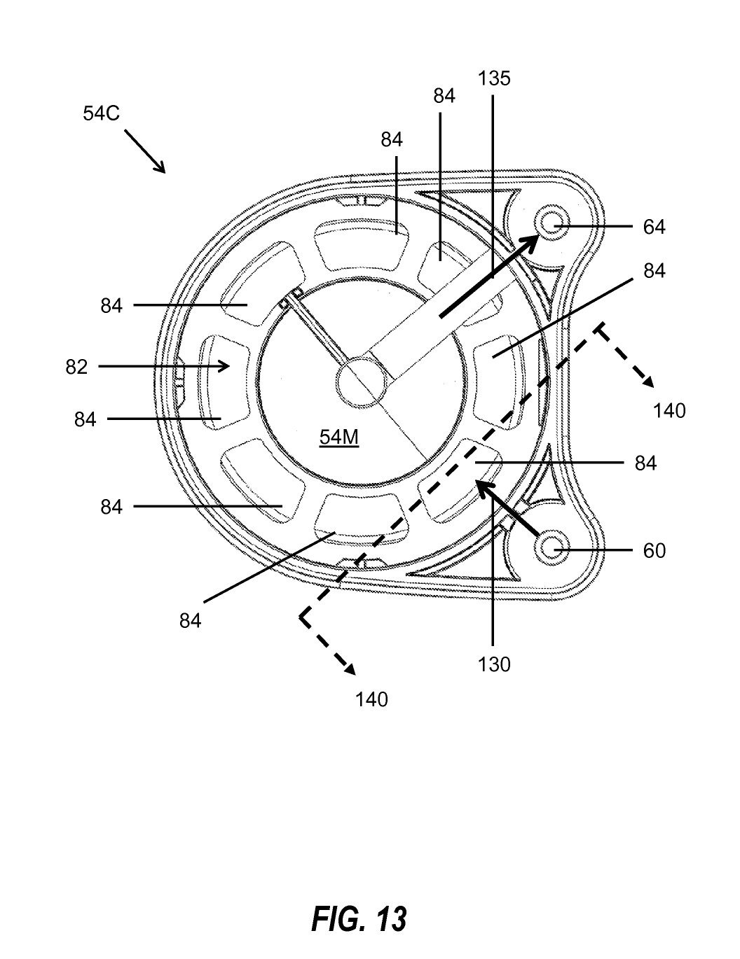

FIG. 13 is a bottom view of another saliva collection reservoir top 54C comprising a bubble and foam collapsing antechamber, according to many embodiments. The saliva collection reservoir top 54C can be similar in many ways to the saliva collection reservoir 54B described above. Unlike the saliva collection reservoir 54B in which the antechamber 82 is open at its floor through the openings 84 distributed over the floor, the antechamber 82 of the saliva collection reservoir top 54C is open at its floor through the openings 84 which are distributed along the periphery of the floor in an annular configuration.

As shown in FIG. 13, the flow of air and saliva can enter the antechamber 82 through inlet valve 60 as shown by arrow 130. The flow of air can then pass through a membrane assembly 54M before exiting the reservoir 50 through outlet valve 64 as shown by arrow 135. The membrane assembly 54M may include and/or accommodate membrane or barrier 40 described above. Referring back to the entry of air and saliva, the saliva/air mix can enter the antechamber 82 from the inlet valve 60 and in many cases blows bubbles. The height of the bubbles entering the antechamber 82 is often limited by the top lid surface of the top 54C and the open floor on the bottom. The bubbles, limited in this way, blow into the antechamber 82. As the bubbles enlarge, the height limitations, often in combination with the large floor openings 84, can increase the stress on the bubbles to a point that the bubbles burst. The saliva of the bubble can then drop into the saliva collection reservoir 50. Thus, the antechamber 82 can stress the bubbles to bursting before they enter the main chamber of the reservoir 50.

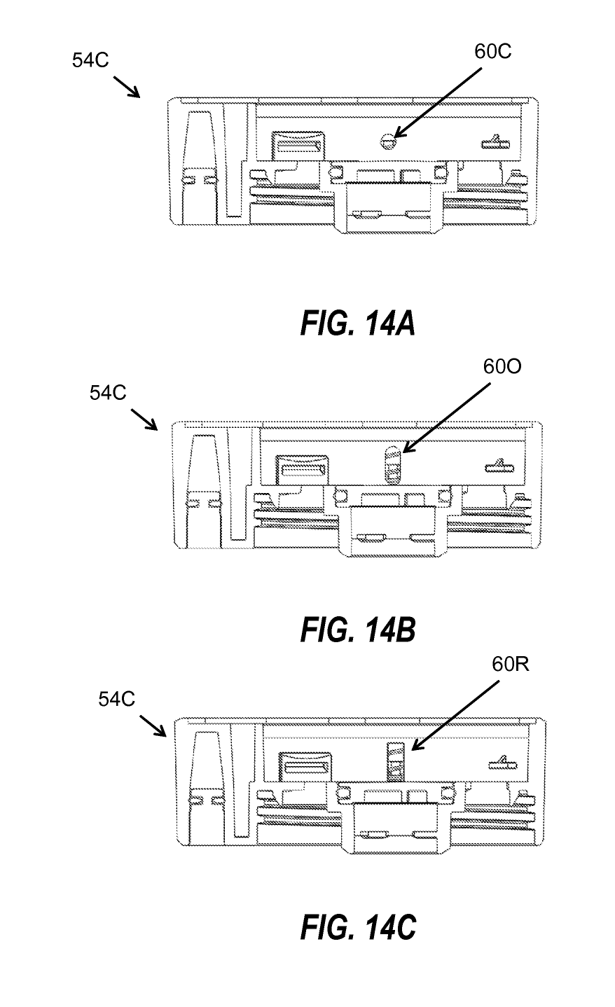

FIGS. 14A to 14C show sectional views of the saliva collection reservoir 54C of FIG. 13 taken from line 140 in FIG. 13. The inlet through which the saliva/air mix enters the antechamber 82 can have many shapes, including but limited to a circle 60C (FIG. 14A), an ellipse, an oval 60O (FIG. 14B), a triangle, a rectangle 60R (FIG. 14C), a square, combinations thereof, and the like. The shape of the inlet can be selected to promote the bursting of any entering saliva bubbles.

While preferred embodiments of the present disclosure have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the disclosure described herein may be employed in practicing the disclosure. It is intended that the following claims define the scope of the invention and that methods and structures within the scope of these claims and their equivalents be covered thereby.

What is claimed is:

1. A saliva collector for attachment in a vacuum line which aspirates an air stream entrained with saliva, said saliva collector comprising:
a reservoir having a bottom, a top, and a sidewall which together defines an interior volume, said reservoir further having an air inlet and an air outlet with an air flow path therebetween,
wherein the top of the reservoir comprises an antechamber positioned along the air flow path and having an interior volume shaped to promote the collapse of bubbles and foam present in the air stream before such bubbles or foam can exit from the outlet.

2. A saliva collector as in claim 1, wherein the air inlet is oriented transverse to gravity to guide the aspirated air to the interior volume of the antechamber.

3. A saliva collector as in claim 2, wherein the air inlet has a shape selected from a group comprising a circle, an oval, an ellipse, a triangle, a rectangle, a square, and combinations thereof.

4. A saliva collector as in claim 1, wherein the antechamber comprises one or more openings configured to allow collapsed bubbles and foam to drop or drain into the interior volume of the reservoir.

5. A saliva collector as in claim 2, wherein the one or more openings of the antechamber open downward toward the interior volume of the reservoir.

6. A saliva collector as in claim 1, further comprising a barrier separating the reservoir into an outer reservoir portion and an inner reservoir portion.

7. A saliva collector as in claim 6, wherein the barrier has a cylindrical shape.

8. A saliva collector as in claim 6, wherein the air flow path crosses the barrier.

9. A method for removing saliva from an air stream aspirate from a patient's oral cavity, said method comprising:
directing the air stream through a reservoir from an inlet, along a flow path, and to an outlet, wherein saliva entrained in the air stream can form bubbles and foam; and
passing the air stream through an antechamber positioned along the flow path to cause bubbles and foam to collapse to provide a pre-treated air stream before passing out through the outlet, wherein the antechamber has an interior volume shaped to promote the collapse of bubbles and foam present in the air stream.

10. A method as in claim 9, wherein the air inlet is oriented transverse to gravity to guide the aspirated air to the interior volume of the antechamber.

11. A method as in claim 10, wherein the air inlet has a shape selected from a group comprising a circle, an oval, an ellipse, a triangle, a rectangle, a square, and combinations thereof.

12. A method as in claim 9, further comprising allowing collapsed bubbles and foam to drop or drain into the interior volume of the reservoir through one or more openings of the antechamber.

13. A method as in claim 12, wherein the one or more openings of the antechamber open downward toward the interior volume of the reservoir.

14. A method as in claim 9, wherein the reservoir includes a barrier separating the reservoir into an outer reservoir portion and an inner reservoir portion.

15. A method as in claim 14, wherein the barrier has a cylindrical shape.

16. A method as in claim 14, wherein the air stream is directed across the barrier.
